(19) United States
(12) Patent Application Publication
(10) Pub. No.: US 2024/0016499 A1
(43) Pub. Date: Jan. 18, 2024

(54) BRAIDED IMPLANT WITH ATRAUMATIC END

(71) Applicant: DePuy Synthes Products, Inc., Raynham, MA (US)

(72) Inventors: Ruijiao XU, Miami Lakes, FL (US); Lacey GOROCHOW, Miami, FL (US); Patrick BROUWER, Raynham, MA (US)

(73) Assignee: DePuy Synthes Products, Inc., Raynham, MA (US)

(21) Appl. No.: 17/866,675

(22) Filed: Jul. 18, 2022

(51) Int. Cl. A61B 17/12

(52) U.S. Cl. CPC A61B 17/12172; A61B 17/12113; A61B 2017/1205; A61B 2017/00526

(57) ABSTRACT

An occlusive device can include a tubular braid including an open end and a pinched end. The tubular braid can define a longitudinal axis. In a predetermined shape, the tubular braid can include an outer sack and an inner sack. The open end can include a plurality of braided wires that are deflected towards the longitudinal axis in the predetermined shape.

### FIELD OF INVENTION

The present invention generally relates to medical instruments, and more particularly, to embolic implants for aneurysm therapy.

### BACKGROUND

Cranial aneurysms can be complicated and difficult to treat due to their proximity to critical brain tissues. Recently, tubular braided implants have been introduced that have the potential to treat an aneurysm or other arterio-venous malformation easily, accurately, and safely in a parent vessel without blocking flow into perforator vessels communicating with the parent vessel. Implant devices for treating aneurysms must be delivered through long, small, tortuous blood vessels and positioning must be controlled precisely to ensure aneurysm filling without causing additional occlusions or clotting in nearby vessels. Additionally, implant devices must be able to be deployed to occlude an aneurysm without causing trauma to the surrounding vessels. Accordingly, there is a need for improved methods, devices, and systems for providing a braided implant with an atraumatic end for occluding aneurysms.

### SUMMARY

It is an object of the present invention to provide systems, devices, and methods to meet the above-stated needs. Generally, it is an object of the present invention to provide an occlusive device for occluding an approximately spherical cavity. The occlusive device can include a tubular braid including an open end and a pinched end. The tubular braid can define a longitudinal axis. In a predetermined shape, the tubular braid can include an outer sack and an inner sack. The open end can include a plurality of braided wires deflected towards the longitudinal axis in the predetermined shape.

In some examples, the tubular braid can include a deployed configuration when deployed within the approximately spherical cavity such that the deployed configuration is based at least in part on the predetermined shape as constrained by the approximately spherical cavity. The plurality of braided wires of the open end can include a distal atraumatic segment and an atraumatic inversion such that the atraumatic inversion inhibits the braided wire ends of the open end from having direct contact with the approximately spherical cavity in the deployed configuration.

In some examples, the plurality of braided wires are deflected towards a proximal end of the tubular braid in the predetermined shape.

In some examples, the plurality of braided wires are deflected orthogonal to the longitudinal axis in the predetermined shape.

In some examples, the tubular braid can include a deployed configuration when deployed within the approximately spherical cavity such that the deployed configuration is based at least in part on the predetermined shape as constrained by the approximately spherical cavity. In the deployed configuration, the inner sack can be configured to expand against the outer sack and urge the outer sack against an inner wall of the approximately spherical cavity.

In some examples, in the predetermined shape, the outer sack can include a distal atraumatic segment extending from the open end and an outer segment extending from the distal atraumatic segment to a first inversion. The inner sack can include a middle segment encircled by the outer segment and extending from the first inversion to a second inversion and an inner segment surrounded by the middle segment and extending from the second inversion to the pinched end.

In some examples, in the predetermined shape, the distal atraumatic segment can include an atraumatic inversion and the open end is positioned in a proximal direction in relation to the atraumatic inversion.

In some examples, in the predetermined shape, the tubular braid can include a distal inversion at a distal end of the inner sack, and wherein the outer sack extends across the distal inversion deflecting proximally towards the longitudinal axis.

In some examples, the open end can include a fold facing in a proximal direction such that the fold forms an atraumatic inversion.

In some examples, in a non-deployed configuration the tubular braid is configured to traverse a lumen of a microcatheter.

In some examples, in the non-deployed configuration the tubular braid can include a length measurable from the pinched end to the open end.

In another aspect, a method of occluding an approximately spherical cavity is disclosed. The method can include delivering a tubular braid including an open end including a plurality of braided wires and a pinched end. The tubular braid can define a longitudinal axis through a vasculature to the approximately spherical cavity while the tubular braid is in a non-deployed configuration. The method can include deploying the tubular braid into a deployed configuration by distally pushing the tubular braid into the approximately spherical cavity whereby the tubular braid expands to form an outer sack. The method can include further distally pushing the tubular braid into the approximately spherical cavity whereby the tubular braid expands to form an inner sack. The method can include positioning the outer sack in the approximately spherical cavity such that the plurality of braided wires of the open end are deflected towards the longitudinal axis. The method can include positioning the inner sack in the approximately spherical cavity such that the inner sack exerts a force against a wall of the approximately spherical cavity and the outer sack. The method can include deflecting, diverting, or slowing flow into the approximately spherical cavity when the outer sack is positioned within the approximately spherical cavity and the inner sack is positioned within the outer sack.

In some examples, the plurality of braided wires of the open end can include a distal atraumatic segment and an atraumatic inversion such that the atraumatic inversion is positioned to inhibit ends of the plurality of wires of the open end from having direct contact with the approximately spherical cavity while the tubular braid is in the deployed configuration.

In some examples, the plurality of braided wires are deflected towards a proximal end of the tubular braid while the tubular braid is in the deployed configuration.

In some examples, the plurality of braided wires are deflected orthogonal to the longitudinal axis while the tubular braid is in the deployed configuration.

In some examples, in a non-deployed configuration, the tubular braid can include a length measurable from the pinched end to the open end measuring between approximately 10 mm and approximately 40 mm.

In another aspect, a method of forming an occlusive device is disclosed. The method can include forming a tubular braid that includes a distal open end and a proximal pinched end, the tubular braid defining a longitudinal axis. The method can include shaping the occlusive device to a predetermined shape to which the occlusive device is capable of self-expanding. The shaping can include a forming a first portion of the tubular braid to a substantially concave shape extending distally with the distal open end. The distal open end can include a plurality of wires deflected towards the longitudinal axis when the occlusive device is in a predetermined shape. The shaping can include inverting a second portion of the tubular braid to form a proximal inversion facing towards a distal direction to define an outermost section of the second braided segment. The forming can include inverting the second portion of the tubular braid to form a distal inversion facing towards the proximal direction to form a middle section between the proximal and distal inversion of the second braided segment that defines an innermost section between the distal inversion and the pinched end.

In some examples, the method can include collapsing the occlusive device and positioning the occlusive device in a microcatheter sized to traverse neurovasculature.

In some examples, the method can include affixing a delivery system to the occlusive device approximate the pinched end such that the pinched end can be manipulated to move the occlusive device from a distal end of the microcatheter.

In some examples, the method can include deflecting the plurality of braided wires orthogonally to the longitudinal axis in the predetermined shape.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example implant having an atraumatic end, according to aspects of the present invention;

FIG. 2 is an illustration of an example implant with braided wires of the atraumatic end having an angle of deflection towards a centerline axis, according to aspects of the present invention;

FIG. 3A is an illustration of an alternate embodiment of an example implant having an atraumatic end, according to aspects of the present invention;

FIG. 3B is an illustration of a partially deployed implant having an atraumatic inversion, according to aspects of the present invention;

FIG. 4 is an illustration of measurements of an example aneurysm, according to aspects of the present invention;

FIG. 5 is an illustration of an example implant in a non-deployed configuration as it is delivered to a treatment site through a microcatheter, according to aspects of the present invention;

FIGS. 6A and 6B are illustrations of an example implant attached to a delivery tube, according to aspects of the present invention;

FIGS. 7A and 7B are illustrations of a detachment sequence of the example implant from the delivery tube, according to aspects of the present invention;

FIG. 8 is a flowchart of a method for occluding a spherical cavity, according to aspects of the present invention; and

FIG. 9 is a flowchart of a method for constructing an occlusive device, according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

Examples presented herein generally include a braided implant that can secure within an aneurysm sac and occlude a majority of the aneurysm's neck. The implant can include a tubular braid that can be set into a predetermined shape, compressed for delivery through a microcatheter, and implanted in at least one implanted position that is based on the predetermined shape and the geometry of the aneurysm in which the braid is implanted. When compressed, the implant can be sufficiently short to mitigate friction forces produced when the implant is delivered unsheathed through the microcatheter allowing for a more simplistic delivery system compared to some other known braided embolic implant delivery systems. The implant can include compatible features, and be constructed or implanted by compatible means, as understood by a person skilled in the pertinent art as described in U.S. Pat. No. 10,653,425, the entirety of which is incorporated herein by reference as if included in full.

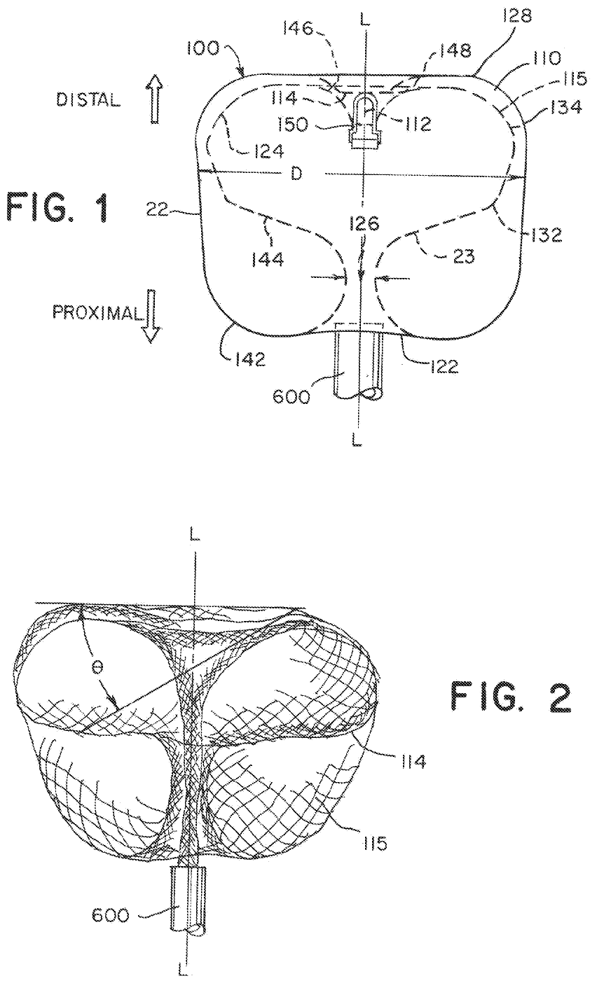

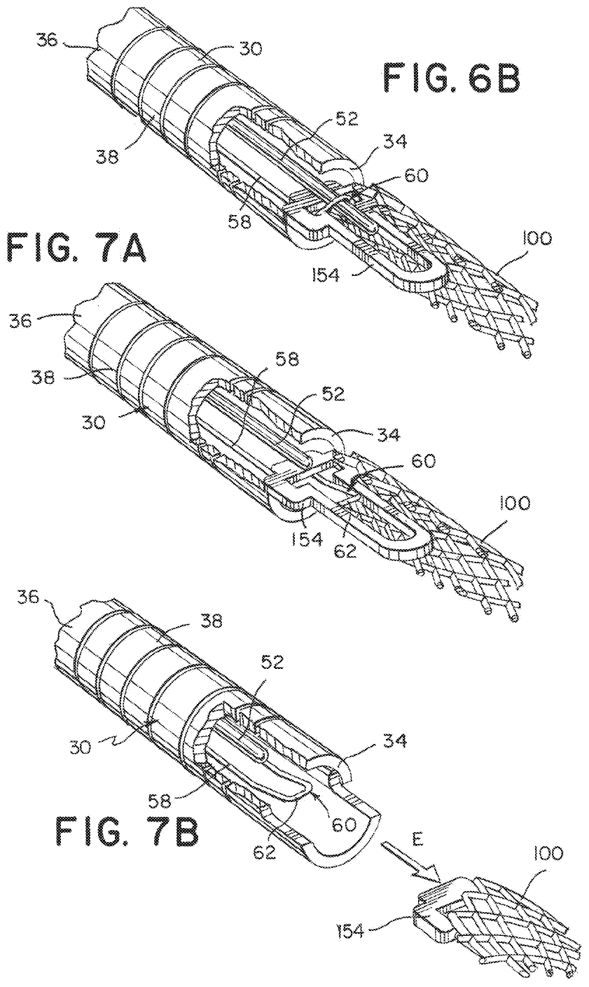

FIG. 1 illustrates an example occlusive device 100 having an atraumatic end, according to aspects of the present invention. The occlusive device 100 is shown in a deployed configuration having been delivered through vasculature by microcatheter 600. The occlusive device 100 can form a tubular braid 110 that includes a pinched end 112 that is attached to a detachment feature 150. Detachment feature 150 can be detachably attached to a delivery tube 30, as will be described in more detail with respect to FIGS. 6A-6B and FIGS. 7A-7B. The tubular braid 110 can include an open end 114 positioned near a distal end of the tubular braid 110 in the predetermined shape. As illustrated, the open end 114 can be deflected such that wire ends of the open end 114 are angled away from the distal end of the tubular braid 110 in the predetermined shape. The deflection of the open end 114 in the predetermined shape can inhibit wire ends of the open end 114 from directly contacting walls of the aneurysm as the implant 100 is deployed.

In a deployed configuration, the occlusive device 100 can be inverted out of microcatheter to form an inner sack 23 and an outer sack 22. Outer sack 22 can include a distal atraumatic segment 148 extending from the open end 114. The outer sack 22 can also include an outer segment 142 extending from the distal atraumatic segment 148 towards a proximal end of occlusive device 100. Proximal of the distal atraumatic segment 148 can be a first inversion 122. The inner sack 23 can include a middle segment 144 that can be encircled by the outer segment 142. Inner sack 23 can extend from the first inversion 122 to a second inversion 124. The inner sack 23 can also include an inner segment 146 that is surrounded by the middle segment 144 and extends from the second inversion 124 towards the pinched end 112.

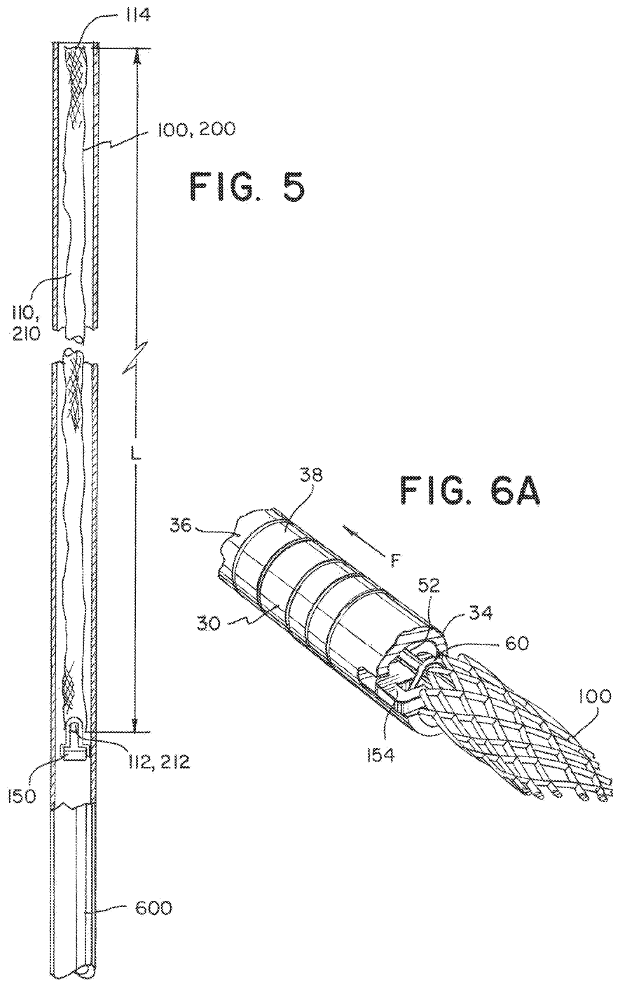

In a non-deployed configuration, occlusive device 100 have a tubular braided configuration, as described in more detail with respect to FIG. 5.

In some examples, the atraumatic distal segment 148 can form an atraumatic inversion 128. Atraumatic distal segment 148 can be angled towards a longitudinal axis L-L in a proximal direction, which allows an atraumatic bend of the occlusive device 100 to interface to minimize trauma to tissue when occlusive device 100 is delivered to and deployed at a treatment site. The tubular braid 110 can include bend 132 and bend 134 that facilitate inversion of the tubular braid 110 in the deployed configuration.

In some examples, the tubular braid has a predetermined shape when expanded out of the microcatheter 600, that can be heat seat. The deployed configuration of the tubular braid 110 can be based at least in part on the predetermined shape as constrained by a treatment site, such as a spherical cavity (shown below in FIG. 4), which in some embodiments can be an aneurysm. In some examples, in the deployed configuration, the inner sack 23 can be configured to expand against the outer sack 22 and urge the outer sack 22 against an inner wall of a spherical cavity. In some examples, the tubular braid can include a distal inversion 124 at a distal end of the inner sack, and the outer sack 22 can be configured to extend across the distal inversion 124 and deflect proximally towards the longitudinal axis.

FIG. 2 illustrates an example occlusive device with braided wires of the atraumatic end having an angle of deflection towards a centerline axis (e.g., longitudinal axis L-L). As shown, the occlusive device 100 can be constructed of a plurality of braided wires 115. The plurality of braided wires 115 can form the open end 114, and can be deflected towards the longitudinal axis L-L to form an atraumatic distal segment 148. The angle of deflection θ of the braided wires 115 can be zero degrees to be orthogonal to the longitudinal axis L-L (e.g., 90 degrees offset from longitudinal axis L-L), while in other examples, the braided wires 115 can be defected towards the proximal end of the occlusive device. In some examples, the angle of deflection θ can be up to approximately 60 degrees (e.g., 30 degrees offset from longitudinal axis L-L).

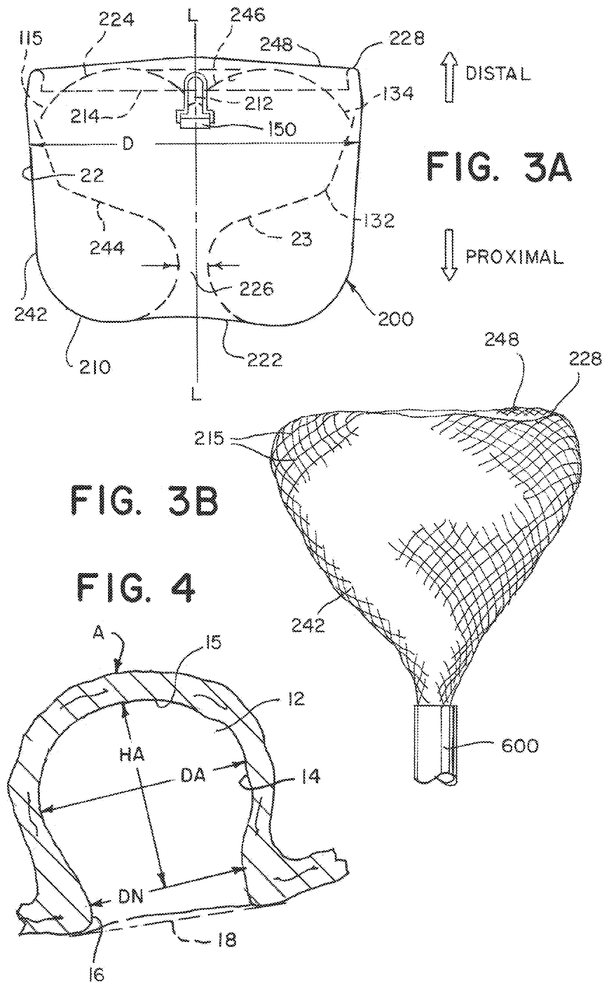

FIG. 3A illustrates an alternate embodiment of an example occlusive device having an atraumatic end. FIG. 3A shows occlusive device 200 in a predetermined shape. Occlusive device 200 may be similar to the occlusive device 100 illustrated in FIGS. 1 and 2, except that an atraumatic distal segment 248 near the open end 214 of occlusive device 200 in FIG. 3A is folded proximally to define an atraumatic inversion 228 that has a smaller radius of curvature as compared to the more gradual bend of atraumatic inversion 128 of occlusive device 100 in FIGS. 1 and 2. While the atraumatic distal segment 148 of the device 100 in FIGS. 1 and 2 extend across the distal inversion 124 of the inner sac 23, the atraumatic distal segment 248 of the occlusive device 200 illustrated in FIG. 3A does not extend across a distal inversion 224 of an inner sac 23. In some examples, the atraumatic distal segment 248 can encircle the distal inversion 224. In some examples, the atraumatic inversion 228 can have a diameter that is greater than a diameter of the distal inversion 224. The atraumatic inversion 228 can inhibit ends of wires of the braid 210 from directly contacting walls of a spherical cavity, such as an aneurysm, when the braid 210 is deployed into the spherical cavity.

The occlusive device 200 can include a tubular braid 210 that includes features similar to corresponding features of the tubular braid 110 illustrated in FIGS. 1 and 2 including a pinched end 212, an outer segment 242, a middle segment 244, an inner segment 246, a first/proximal inversion 222, a second/distal inversion 246, and bends 132, 134. The occlusive device 200 can include a detachment feature 150 configured as described in more detail with respect to FIGS. 6A-6B and FIGS. 7A-7B. In a deployed configuration, the occlusive device 200 can be inverted out of microcatheter to form an inner sack 23 and an outer sack 22 similar to as described in relation to FIGS. 1 and 2. Outer sack 22 can include a distal atraumatic segment 248 extending from the open end 214. The outer sack 22 can also include an outer segment 242 extending from the distal atraumatic segment 248 towards a proximal end of occlusive device 200. Proximal of the distal atraumatic segment 248 can be a proximal inversion 222. The inner sack 23 can include a middle segment 244 that can be encircled by the outer segment 142. Inner sack 23 can extend from the proximal inversion 222 to a distal inversion 224. The inner sack 23 can also include an inner segment 246 that is surrounded by the middle segment 244 and extends from the distal inversion 224 towards the pinched end 212.

In a non-deployed configuration, occlusive device 200 have a tubular braided configuration, as described in more detail with respect to FIG. 5.

In some examples, the atraumatic distal segment 248 can form an atraumatic inversion 228. Atraumatic distal segment 248 can be angled towards a longitudinal axis L-L in a proximal direction, which allows an atraumatic bend of the occlusive device 200 to interface to minimize trauma to tissue when occlusive device 200 is delivered to and deployed at a treatment site.

In some examples, the tubular braid 210 has a predetermined shape when expanded out of the microcatheter 600, that can be heat seat. The deployed configuration of the tubular braid 210 can be based at least in part on the predetermined shape as constrained by a treatment site, such as a spherical cavity (shown below in FIG. 4), which in some embodiments can be an aneurysm. In some examples, in the deployed configuration, the inner sack 23 can be configured to expand against the outer sack 22 and urge the outer sack 22 against an inner wall of a spherical cavity. The tubular braid 210 can include bend 132 and bend 134 that facilitate inversion of the tubular braid 210 in the deployed configuration.

FIG. 3B illustrates a partially deployed occlusive device 200 having an atraumatic inversion 228. Occlusive device 200 can take the partially deployed configuration just after the tubular braid 210 exits microcatheter 600 and before the tubular braid 210 begins to invert upon itself to form the outer segment 242, middle segment 244, and inner segment 246. As seen in FIG. 3B, the braided wires 215 can form the atraumatic distal segment 248 that has an atraumatic inversion 228. The atraumatic inversion 228 can be formed by the braided wires 215 being deflected towards a centerline axis of the occlusive device 200 (e.g., longitudinal axis L-L). The deflection of the braid wires 215 near the open end 214 can inhibit ends of the braid wires 215 from directly contacting walls of a spherical cavity, such as an aneurysm, when the braid 210 is deployed into the spherical cavity.

FIG. 4 illustrates measurements of an example spherical cavity A. FIG. 4 illustrates height HA, sac diameter DA, and neck diameter DN measurements of a spherical cavity A. In some examples, spherical cavity can be an aneurysm of a patient. The location of the plane 18 defining a boundary between the spherical cavity A and blood vessels is also illustrated. As shown, the spherical cavity A can include a neck 16, a wall 14, and a distal wall 15. The spherical cavity A can also include a spherical cavity interior 12. The occlusive device 100 and/or 200 can be configured to occlude the spherical cavity A when the occlusive device is in a deployed configuration. As described above, the deployed configuration of occlusive device 100, 200 can be based at least in part on the geometry of spherical cavity A as well as the predetermined heat-set shape of occlusive device 100, 200. Accordingly, occlusive device 100, 200 can be configured to fit spherical cavities A of various geometries (e.g., varying proportions of sac diameter DA, height HA, and neck diameter DN).

When deployed, the braids 110, 210 of example occlusive devices 100, 200 illustrated herein can extend across the neck 16 of the aneurysm A to block flow of blood into the aneurysm A. The atraumatic segment 148, 248 can be positioned near the distal wall 15 of the cavity and the proximal/first inversion 122, 222 can be positioned near the neck 16. Preferably, the diameter D of the braid 110, 210 in the predetermined shape (FIGS. 1, 2, and 3A) is greater than the sac diameter DA of the aneurysm A to be treated so that as the braid 110, 210 moves to the predetermined shape while confined with the aneurysm A, the sacks 22, 23 of the braid 110, 210 press into the wall 14 of the aneurysm A.

FIG. 5 is an illustration of an example occlusive device in a non-deployed configuration as it is delivered to a treatment site through a microcatheter. As shown, the occlusive device 100, 200 can be in a non-deployed (e.g., collapsed) configuration as it is delivered to a treatment site (e.g., spherical cavity A) through a microcatheter 600. As shown, occlusive device 100, 200 be formed of a tubular braid 110, 210, which can be sized to have a length L. The illustrated occlusive device 100, 200 can have a length L of between about 22 mm and about 25 mm. In other examples, occlusive device 100, 200 can have a length between approximately 10 mm and approximately 40 mm. As will be appreciated and understood by a person of ordinary skill in the art, the length L of a specific tubular braid 110, 210 can be tailored based on the size and shape of the aneurysm being treated.

FIGS. 6A and 6B are illustrations of an example occlusive device attached to a delivery tube. FIG. 6A shows delivery tube 30 attached to occlusive device 100 via a pull wire 52, loop wire 58, and locking portion 154 of detachment feature 150. The delivery tube can include a distal end 34, proximal end 36, and compressible portion 38. The detachment feature 150 can be positioned just distal of the delivery tube 30 and a proximal end of the occlusive device 100, 200 can be attached to a distal end of the detachment feature 150. A pull wire 52 can be positioned within a lumen of the delivery tube 30. A loop wire 58 can be attached to the delivery tube 30 and can include a loop opening 60 at a distal end 34 of the delivery tube 30. As shown more clearly in FIG. 3B, which is a cutaway view of the delivery tube of FIG. 3A, the loop opening 60 can be passed through the locking portion 154 of the detachment mechanism 50, and a distal end of the pull wire 52 can be positioned through the loop opening 60 of the loop wire 58 after it is passed through the locking portion 154. Accordingly, the detachment feature 150 can thus be attached to the distal end 34 of the delivery tube 30. The compressible portion 38 can be formed of spiral cuts within the material of the delivery tube 30, such that the compressible portion 38 can be compressed prior to attaching the detachment mechanism 150 to the delivery tube 30. When the occlusive device 100, 200 is detached from the delivery tube, the compressible portion 38 can be configured to expand and impart an elastic force to “push” the occlusive device 100, 200 away from the deployment tube 30 and towards the treatment site.

FIGS. 7A and 7B are illustrations of a detachment sequence of the example occlusive device from the delivery tube. In FIG. 7A, the pull wire 52 is shown being pulled proximally towards proximal end 36 of the delivery tube 30. This allows distal end of pull wire 52 to exit the loop opening 60 of loop wire 58, thereby allowing loop opening 60 to exit the locking portion 154 of detachment feature 150. FIG. 7A shows loop opening distal end 62 of loop opening exiting the locking portion 154 of detachment feature 150. In FIG. 7B, the compressible portion 38 of delivery tube 30 can decompress, thereby imparting elastic force “E” to the detachment feature 150.

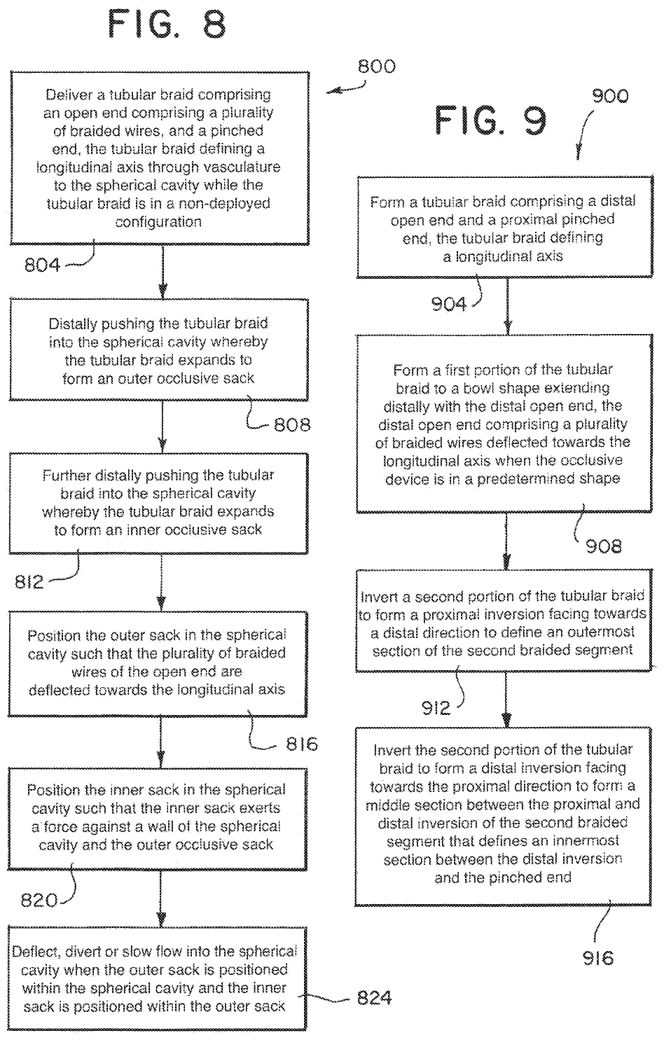

FIG. 8 is a flowchart of a method 800 for occluding a spherical cavity. In block 804, the method can include delivering a tubular braid 110, 210 defining a longitudinal axis L-L through vasculature to the approximately spherical cavity (A) while the tubular braid is in a non-deployed configuration. The tubular braid can include an open end 114, 214 that includes a plurality of braided wires 115, 215 and a pinched end 112, 212.

In block 808, the method can include distally pushing the tubular braid 110, 210 into the approximately spherical cavity A whereby the tubular braid 110, 210 can expand to form an outer sack 22.

In block 812, the method can include further distally pushing the tubular braid 110, 210, into the approximately spherical cavity A whereby the tubular braid (110, 210) expands to form an inner sack 23.

In block 816, the method can include positioning the outer sack 22 in the approximately spherical cavity A such that the plurality of braided wires 115, 215 of the open end 114, 214 are deflected towards the longitudinal axis L-L.

In block 820, the method can include positioning the inner sack 23 in the approximately spherical cavity A such that the inner sack 23 exerts a force against a wall of the approximately spherical cavity A and the outer sack 22.

In block 824, the method can include deflecting, diverting, or slowing flow into the approximately spherical cavity A when the outer sack 22 is positioned within the approximately spherical cavity A and the inner sack 23 is positioned within the outer sack 22.

FIG. 9 is a flowchart of a method 900 for constructing an occlusive device. In block 904, the method can include forming a tubular braid 110, 210 that includes a distal open end 114, 214 and a proximal pinched end 112, 212. The tubular braid 110, 210 can define a longitudinal axis L-L. The method can include shaping the occlusive device 100, 200 to a predetermined shape to which the occlusive device is capable of self-expanding.

In some examples, the plurality of braided wires 115, 215 of the open end 114, 214 can include a distal atraumatic segment 148, 248 and an atraumatic inversion 128, 228. In some examples, only the atraumatic inversion is configured to have direct contact with the approximately spherical cavity A while the tubular braid 110, 210 is in the predetermined shape.

In some examples, the plurality of braided wires 115, 215 can be deflected towards a proximal end of the tubular braid 110 while the tubular braid 110 is in the predetermined shape.

In some examples, the plurality of braided wires 115, 215 are deflected along an orthogonal axis O-O that is orthogonal to the longitudinal axis L-L while the tubular braid 110, 210 is in the predetermined shape.

In some examples, in a non-deployed configuration, the tubular braid 110, 210 can have a length L measurable from a pinched end 112, 212 to the open end 114, 214 measuring between approximately 10 mm and 40 mm.

In block 908, shaping the occlusive device can include forming a first portion of the tubular braid 110, 210 to a substantially concave shape extending distally with the distal open end 114, 214. The distal open end 114, 214 can include a plurality of braided wires 115, 215 deflected towards the longitudinal axis L-L when the occlusive device is in a predetermined shape.

In block 912, shaping the occlusive device can include inverting a second portion of the tubular braid 110, 210 to form a proximal inversion facing towards a distal direction to define an outermost section of the second braided segment.

In block 916, shaping the occlusive device can include inverting the second portion of the tubular braid 110, 210 to form a distal inversion. The distal inversion can face towards the distal direction to form a middle section between the proximal inversion and the distal inversion of the second braided segment. The distal inversion can define an innermost section between the distal inversion and the pinched end.

In some examples, the method can include collapsing the occlusive device 100, 200, and positioning the occlusive device 100, 200 in a microcatheter sized to traverse neurovasculature.

In some examples, the method can include affixing a delivery system to the occlusive device 100, 200 approximate the pinched end 114, 214 such that the pinched end 114, 214 can be manipulated to move the occlusive device 100, 200 from a distal end of the microcatheter 600.

In some examples, the method can include deflecting the plurality of braided wires 115, 215 orthogonally to the longitudinal axis L-L in the predetermined shape.

The example occlusive device 100, 200 described herein can rely on a radial outward force to anchor the occlusive device within the sac of an aneurysm. To this end, the tubular braid 110, 210 can be shaped to a predetermined shape having a diameter that is greater than its height so that the braid is radially constricted when implanted in an aneurysm. The ratio of diameter to height of the braid 110, 210 in a respective predetermined shape can be within the range of 2:1 to 1:3 to treat aneurysms of many known sizes and shapes.

The descriptions contained herein are examples of embodiments of the invention and are not intended in any way to limit the scope of the invention. As described herein, the invention contemplates many variations and modifications of the occlusive device, including alternative materials, alternative geometries, alternative detachment features, alternative delivery systems, alternative means for forming a braid into a predetermined shape, alternative treatment methods, etc. These modifications would be apparent to those having ordinary skill in the art to which this invention relates and are intended to be within the scope of the claims which follow.

What is claimed is:

1. An occlusive device for occluding an approximately spherical cavity, comprising:
a tubular braid comprising an open end and a pinched end, the tubular braid defining a longitudinal axis,
wherein, in a predetermined shape, the tubular braid comprises an outer sack and an inner sack, and
wherein, the open end comprises a plurality of braided wires deflected towards the longitudinal axis in the predetermined shape.

2. The occlusive device of claim 1,
wherein the tubular braid comprises a deployed configuration when deployed within the approximately spherical cavity such that the deployed configuration is based at least in part on the predetermined shape as constrained by the approximately spherical cavity, and
wherein the plurality of braided wires of the open end comprise a distal atraumatic segment and an atraumatic inversion such that the atraumatic inversion is configured to inhibit ends of the plurality of braided wires at the open end from having direct contact with the approximately spherical cavity in the deployed configuration.

3. The occlusive device of claim 1, wherein the plurality of braided wires are deflected towards a proximal end of the tubular braid in the predetermined shape.

4. The occlusive device of claim 1, wherein the plurality of braided wires are deflected orthogonal to the longitudinal axis in the predetermined shape.

5. The occlusive device of claim 1,
wherein the tubular braid comprises a deployed configuration when deployed within the approximately spherical cavity such that the deployed configuration is based at least in part on the predetermined shape as constrained by the approximately spherical cavity, and
wherein in the deployed configuration, the inner sack is configured to expand against the outer sack and urge the outer sack against an inner wall of the approximately spherical cavity.

6. The occlusive device of claim 1, wherein, in the predetermined shape:
the outer sack comprises a distal atraumatic segment extending from the open end and an outer segment extending from the distal atraumatic segment to a first inversion; and
the inner sack comprises a middle segment encircled by the outer segment and extending from the first inversion to a second inversion and an inner segment surrounded by the middle segment and extending from the second inversion to the pinched end.

7. The occlusive device of claim 6, wherein in the predetermined shape:
the distal atraumatic segment comprises an atraumatic inversion; and
the open end is positioned in a proximal direction in relation to the atraumatic inversion.

8. The occlusive device of claim 1,
wherein, in the predetermined shape, the tubular braid comprises a distal inversion at a distal end of the inner sack, and
wherein the outer sack extends across the distal inversion, deflecting proximally toward the longitudinal axis.

9. The occlusive device of claim 1, wherein the open end comprises a fold facing in a proximal direction such that the fold forms an atraumatic inversion.

10. The occlusive device of claim 1, wherein in a non-deployed configuration the tubular braid is configured to traverse a lumen of a microcatheter.

11. The occlusive device of claim 1,
wherein the tubular braid comprises a non-deployed configuration when being delivered through a catheter to the approximately spherical cavity, and
wherein in the non-deployed configuration the tubular braid comprises a length measurable from the pinched end to the open end.

12. A method of occluding an approximately spherical cavity, comprising:
delivering a tubular braid comprising an open end comprising a plurality of braided wires, and a pinched end, the tubular braid defining a longitudinal axis through vasculature to the approximately spherical cavity while the tubular braid is in a non-deployed configuration;
deploying the tubular braid into a deployed configuration by:
distally pushing the tubular braid into the approximately spherical cavity whereby the tubular braid expands to form an outer sack;
further distally pushing the tubular braid into the approximately spherical cavity whereby the tubular braid expands to form an inner sack;
positioning the outer sack in the approximately spherical cavity such that the plurality of braided wires of the open end are deflected towards the longitudinal axis;
positioning the inner sack in the approximately spherical cavity such that the inner sack exerts a force against a wall of the approximately spherical cavity and the outer sack; and
deflecting, diverting or slowing flow into the approximately spherical cavity when the outer sack is positioned within the approximately spherical cavity and the inner sack is positioned within the outer sack.

13. The method of claim 12, wherein the plurality of braided wires of the open end comprise a distal atraumatic segment and an atraumatic inversion such that the atraumatic inversion is positioned to inhibit ends of the plurality of braided wires of the open end from having direct contact with the approximately spherical cavity while the tubular braid is in the deployed configuration.

14. The method of claim 12, wherein the plurality of braided wires are deflected towards a proximal end of the tubular braid while the tubular braid is in the deployed configuration.

15. The method of claim 12, wherein the plurality of braided wires are deflected orthogonal to the longitudinal axis while the tubular braid is in the deployed configuration.

16. The method of claim 12, wherein in a non-deployed configuration, the tubular braid comprises a length measurable from the pinched end to the open end measuring between approximately 10 mm and approximately 40 mm.

17. A method of forming an occlusive device, comprising:
forming a tubular braid comprising a distal open end and a proximal pinched end, the tubular braid defining a longitudinal axis;
shaping the occlusive device to a predetermined shape to which the occlusive device is capable of self-expanding by:
forming a first portion of the tubular braid to a substantially concave shape extending distally with the distal open end, the distal open end comprising a plurality of braided wires deflected towards the longitudinal axis when the occlusive device is in a predetermined shape;
inverting a second portion of the tubular braid to form a proximal inversion facing towards a distal direction to define an outermost section of the second portion; and
inverting the second portion of the tubular braid to form a distal inversion facing towards a proximal direction to form a middle section between the proximal inversion and the distal inversion of the second portion that defines an innermost section between the distal inversion and the pinched end.

18. The method of claim 17, further comprising:
collapsing the occlusive device; and
positioning the occlusive device in a microcatheter sized to traverse neurovasculature.

19. The method of claim 17, further comprising:
affixing a delivery system to the occlusive device approximate the pinched end such that the pinched end can be manipulated to move the occlusive device from a distal end of the microcatheter.

20. The method of claim 17, further comprising:
deflecting the plurality of braided wires orthogonally to the longitudinal axis in the predetermined shape.
